(19) United States
(12) Patent Application Publication
(10) Pub. No.: US 2005/0107464 A1
(43) Pub. Date: May 19, 2005

(54) Antitumor agent containing lactic acid oligomer mixture

(76) Inventors: Yasukazu Nagato, Kanagawa (JP); Shigeo Takada, Kanagawa (JP); Chieko Murayama, Kanagawa (JP); Mikio Watanabe, Kanagawa (JP); Masahiro Murakami, Osaka (JP)

Correspondence Address: GREENBLUM & BERNSTEIN, P.L.C., 1950 ROLAND CLARKE PLACE, RESTON, VA, 20191, US

(21) Appl. No.: 10/493,051

(22) PCT Filed: Nov. 5, 2002
(86) PCT No.: PCT/JP02/11505

(30) Foreign Application Priority Data
Nov. 6, 2002 (JP) 2001-340680

(51) Int. Cl. A61K 31/365; A61K 31/225

(52) U.S. Cl. 514/450; 514/548

(57) ABSTRACT

An object of the present invention is to provide a novel antitumor agent by evaluating the antitumor effect obtained by a mixture of polylactic acids which is produced by polymerizing lactides in the presence of certain amine compound. The present invention provides an antitumor agent which comprises a mixture of linear and cyclic lactic acid oligomers represented by the following formula (1) or (2): 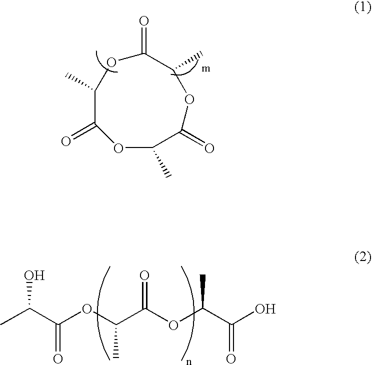 wherein m represents an integer of 1 to 30, and n represents an integer of 1 to 30, which is produced by polymerizing lactides in the presence of a compound represented by the following formula (3):
Me—N(R¹)(R²) (3)
wherein Me represents an alkali metal, and each of R¹ and R² independently represents an aliphatic group or aromatic group.

### TECHNICAL FIELD

The present invention relates to an antitumor agent which comprises a mixture of linear and cyclic lactic acid oligomers. The antitumor agent of the present invention shows tumor reduction effect and metastasis suppression effect, and is useful for the therapy of cancers.

### BACKGROUND ART

A mixture of polylactic acids containing cyclic polylactic acid, which suppresses the anaerobic glycolysis system of cancer cells and exhibits antitumor effects, has been studied, centering on its canceration suppression effect obtained by using a mouse that spontaneously develops cancer, and its tumor growth suppression effect and metastasis suppression effect obtained by using transplanted cancer tissues (Lewis lung cancer cells) (Nagato et al., the 56^{th} conference of the Japanese Cancer Association, September 1997; and Takada et al., the 57^{th} conference of the Japanese Cancer Association, September 1998).

With respect to the mixture of polylactic acids, its chemoprevention effects, an administration method thereof effective through the combined use of anticancer agents or application of radioactive rays, and applied dosages thereof have also been studied. From a high dosage administration experiment, remarkable antitumor effect was not obtained. Under such circumstances, a new attempt to synthesize a new mixture of polylactic acids is being made.

### DISCLOSURE OF THE INVENTION

An object of the present invention is to provide a novel antitumor agent by evaluating the antitumor effect obtained by a mixture of polylactic acids which is produced by polymerizing lactides in the presence of certain amine compound. Another object of the present invention is to provide food and drink prepared by using the antitumor agent as described above.

As a result of intensive studies directed towards achieving the aforementioned objects, the present inventors have carried out an experiment where the antitumor effect of a mixture of polylactic acids (referred to as X03 in the Examples below) which is produced by polymerizing lactides in the presence of certain amine compound is compared with the antitumor effects of mixtures of polylactic acids (referred to as X01 and X02 in the Examples below) which is produced by different methods, and they have succeeded in confirming the utility of the former mixture. Especially, the mixture of polylactic acids according to the present invention showed suppression of tumor weight and showed remarkable suppression of lung metastasis. The present invention has been completed based on these findings.

The present invention provides an antitumor agent which comprises a mixture of linear and cyclic lactic acid oligomers represented by the following formula (1) or (2) (hereinafter also referred to as a mixture of linear and cyclic lactic acid oligomers which is used in the present invention): 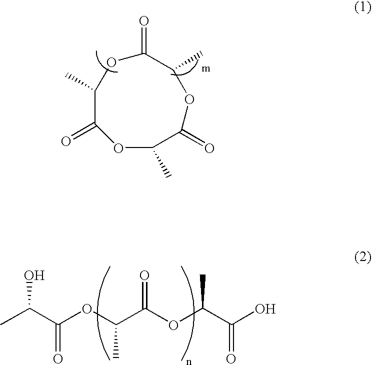
wherein m represents an integer of 1 to 30, and n represents an integer of 1 to 30, which is produced by polymerizing lactides in the presence of a compound represented by the following formula (3):
Me—N(R¹)(R²) (3)
wherein Me represents an alkali metal, and each of R¹ and R² independently represents an aliphatic group or aromatic group.

Preferably, Me is lithium in the formula (3).

Preferably, each of R¹ and R² independently represents an alkyl group containing 1 to 6 carbon atoms in the formula (3).

Preferably, Me is lithium, and each of R¹ and R² is an isopropyl group in the formula (3).

Preferably, m is an integer of 1 to 19 in the formula (1).

Preferably, n is an integer of 1 to 25 in the formula (2).

According to another aspect of the present invention, there is provided food and drink which comprise the antitumor agent according to the invention as described above.

According to further aspect of the present invention, there is provided a use of a mixture of linear and cyclic lactic acid oligomers which is used in the present invention, in the production of the antitumor agent or the food and drink for an antitumor effect.

According to still further aspect of the present invention, there is provided a method for suppressing tumor which comprises administering an effective amount of a mixture of linear and cyclic lactic acid oligomers which is used in the present invention, to mammal animals including human.

### BRIEF DESCRIPTION OF THE DRAWINGS

FIG. 1 shows a mass spectrum of the mixture of polylactic acids obtained in Synthesis Example 1.

FIG. 2 shows an MS spectrum of a reaction product obtained in Synthesis Example 2.

FIG. 3 shows a whole view of an NMR of a reaction product obtained in Synthesis Example 2.

FIG. 4 shows an enlarged view of a part of FIG. 3.

FIG. 5 shows an enlarged view of a part of FIG. 3.

FIG. 6 shows a general view of the positive mode FABMS spectrum of the product obtained in Synthesis Example 3. Range: m/z 10.0000 to 1305.5900

FIG. 7 shows a general view of the negative mode FABMS spectrum of the product obtained in Synthesis Example 3. Range: m/z 10.0000 to 2000.0000

FIG. 8 shows an enlarged view of the negative mode FABMS spectrum of the product obtained in Synthesis Example 3. Range: m/z 10.0000 to 501.9260

FIG. 9 shows an enlarged view of the negative mode FABMS spectrum of the product obtained in Synthesis Example 3. Range: m/z 490.2980 to 1003.7700

FIG. 10 shows an enlarged view of the negative mode FABMS spectrum of the product obtained in Synthesis Example 3. Range: m/z 999.9500 to 1504.3400

FIG. 11 shows an enlarged view of the negative mode FABMS spectrum of the product obtained in Synthesis Example 3. Range: m/z 1484.5300 to 2000.0000

FIG. 12 shows a general view of the NMR spectrum of the product obtained in Synthesis Example 3.

FIG. 13 shows a comparison of measurement results of tumor weights.

FIG. 14 shows a comparison of measurement results of numbers of colony metastasized to a lung and tumor weights.

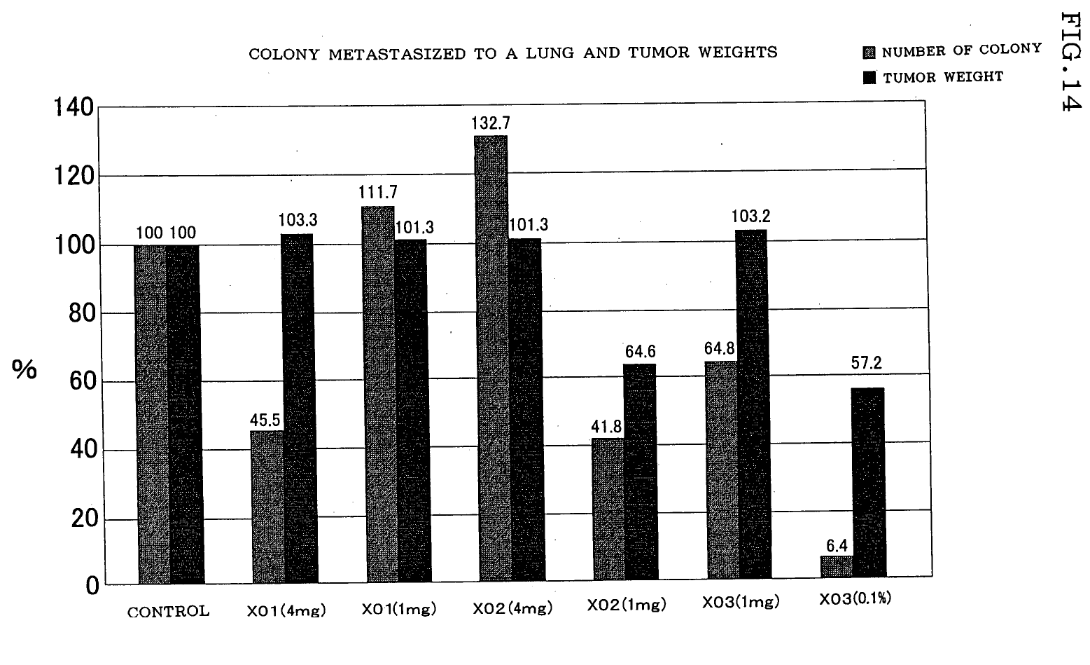

### THE BEST MODE FOR CARRYING OUT THE INVENTION

The methods and embodiments for carrying out the present invention are described in detail below.

The starting material used in the production of the mixture of linear and cyclic lactic acid oligomers which is used as an active ingredient in the antitumor agent and the food and drink according to the present invention, is lactide (3,6-dimethyl-1,4-dioxane-2,5-dione) obtained by condensation of two molecules of lactic acid by dehydration. In the present invention, the lactides are allowed to react in the presence of an alkali metal compound represented by the above formula (3). The formula (3):
Me—N(R¹)(R²) (3)
is explained below.

In the formula (3), Me represents an alkali metal, and each of R¹ and R² independently represents an aliphatic group or aromatic group.

Examples of the aliphatic group defined in the present specification include a straight chain, branched chain, cyclic, or their combined form, saturated or unsaturated aliphatic hydrocarbon group containing 1 to 12, and preferably 1 to 6 carbon atoms. Specific examples include alkyl groups such as methyl, ethyl, n-propyl, i-propyl, n-butyl, i-butyl, t-butyl, octyl and dodecyl, and cycloalkyl groups such as cyclopropyl, cyclobutyl, cyclooctyl and cyclododecyl. The aliphatic group may be an unsaturated hydrocarbon group having a double or triple bond.

Examples of the aromatic group defined in the present invention include an aryl group and an arylalkyl group, containing 6 to 30, preferably 6 to 20, more preferably 6 to 12, and further more preferably 6 to 10 carbon atoms. Examples of the aryl group include phenyl, tolyl and naphthyl, and examples of the arylalkyl group include benzyl, phenethyl and naphthylmethyl.

The aliphatic group and the aromatic group may have one or more substituent(s). The type of substituents is not particularly limited, and the examples include a straight chain, branched chain, linear or cyclic alkyl group, a straight chain, branched chain, linear or cyclic alkenyl group, a straight chain, branched chain, linear or cyclic alkynyl group, an aryl group, an acyloxy group, an alkoxycarbonyloxy group, an aryloxycarbonyloxy group, a carbamoyloxy group, a carbonamide group, a sulfonamide group, a carbamoyl group, a sulfamoyl group, an alkoxy group, an aryloxy group, an aryloxycarbonyl group, an alkoxycarbonyl group, an N-acylsulfamoyl group, an N-sulfamoylcarbamoyl group, an alkylsulfonyl group, an arylsulfonyl group, an alkoxycarbonylamino group, an aryloxycarbonylamino group, an amino group, an ammonio group, a cyano group, a nitro group, a carboxyl group, a hydroxyl group, a sulfo group, a mercapto group, an alkylsulfmyl group, an arylsulfmyl group, an alkylthio group, an arylthio group, an ureide group, a heterocyclic group (e.g., a monocyclic or condensed ring containing at least one or more nitrogen, oxygen or sulfur atom(s) and consisting of 3 to 12 ring forming members), a heterocyclic oxy group, a heterocyclic thio group, an acyl group, a sulfamoylamino group, a silyl group, and a halogen atom. In the above, the carbon number of alkyl, alkenyl, alkynyl and alkoxy is generally 1 to 12, and preferably 1 to 6, and the carbon number of aryl is generally 6 to 20, and preferably 6 to 10.

In the formula (3), Me represents an alkali metal. Examples of an alkali metal include Li, Na and K, and Li is preferred.

Among the compounds represented by the formula (3), the compounds having asymmetric carbon atoms may be any one of (R) form, (S) form, and (R),(S) form.

A method for obtaining an alkali metal compound represented by the formula (3) is not particularly limited, and a person skilled in the art can obtain the compound as appropriate. For example, the alkali metal compound can be obtained by reaction of dialkylamine such as diisopropylamine with an alkylated alkali metal such as n-butyllithium. More specifically, this reaction can be carried out, for example, by mixing a solution containing dialkylamine in an inert solvent such as THF with a solution containing an alkylated alkali metal in an inert solvent such as hexane under conditions that are inactive for the reaction, e.g., under a nitrogen gas atmosphere, and then stirring the mixture. The reaction temperature is not particularly limited, as long as the reaction progresses, but it is preferably −78° C. to room temperature. The reaction temperature can be set as appropriate.

When lactides are polymerized in the presence of a compound represented by the formula (3), the used amount of the compound represented by the formula (3) (Me—N(R¹)(R²)) is preferably 0.1 to 1 mol, and more preferably 0.2 to 0.3 mol per mole of lactide.

When the polymerization reaction of lactides is carried out, the reaction temperature is not particularly limited as long as the reaction progresses, but it is preferably −100° C. to room temperature, and more preferably −78° C. to room temperature.

Polymerization reaction of lactides is preferably carried out in the presence of a reaction solvent. The reaction solvent is not particularly limited as long as it is inactive for the reaction. Examples of preferred solvents include cyclic ethers such as tetrahydrofuran, diethylether, and dimethoxyethane. Examples of reaction atmospheres to be used may include inactive gas atmospheres such as nitrogen gas and argon gas. Reaction pressure is not particularly limited, and it is preferably normal pressure.

The composition of the mixture of linear and cyclic lactic acid oligomers which is obtained by the method as mentioned above is altered depending on the type of the compound of the formula (3) used as a reaction assistant and the reaction conditions. Preferably, the content of linear lactic acid oligomer is higher than that of cyclic lactic acid oligomer.

According to the method as mentioned above, there is produced a mixture of linear and cyclic lactic acid oligomers represented by the following formula (1) or (2): 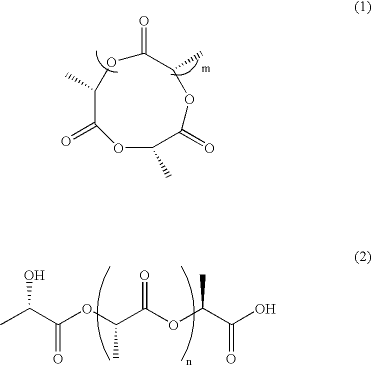
wherein m represents an integer of 1 to 30, and n represents an integer of 1 to 30.

The reaction product is generally a mixture of a cyclic lactic acid oligomer wherein m represents an integer ranging from 1 to 30, e.g., 1 to 28, 1 to 25, 1 to 21, 1 to 19, etc., and a linear lactic acid oligomer wherein n represents an integer ranging from 1 to 30, e.g., 1 to 28, 1 to 25, etc.

When “lactic acid” is simply referred to in the present specification, this lactic acid includes all of L-lactic acid, D-lactic acid or a mixture comprising these types of lactic acid at any ratio. Preferably in the present invention, the lactic acid consists substantially of L-lactic acid. The term “substantially” is used herein to mean that the ratio of L-lactic acid units in a mixture of poly lactic acids (number of L-lactic acid unit/number of L-lactic acid unit+number of D-lactic acid unit×100) is, for example, 70% or more, preferably 80% or more, more preferably 85% or more, further more preferably 90% or more, and particularly preferably 95% or more. The ratio of L-lactic acid units in a mixture of poly lactic acids depends on the ratio of L-lactic acid and D-lactic acid that exist in lactic acids used as a starting substance.

The antitumor agent according to the invention can be widely used for suppressing a tumor. Suppressing the tumor includes prevention of oncogenesis, suppression of tumor enlargement, regression of tumor, and suppression of tumor metastasis, and also includes clinically all of prevention and/or treatment of a cancer and/or a tumor.

The type of the cancer for which the antitumor agent according to the invention can be used is not particularly limited, but includes all of benign tumors and malignant tumors. Examples of the cancers include malignant melanoma, malignant lymphoma, digestive tract cancers, lung cancer, esophagus cancer, stomach cancer, large bowel cancer, rectum cancer, colonic cancer, urinary tract tumor, gallbladder cancer, cholangiocarcinoma, biliary tract cancer, breast carcinoma, hepatic cancer, pancreatic cancer, testis tumor, cancer of the upper jaw, cancer of the tongue, cancer of the lip, cancer of the oral cavity, cancer of pharynx, cancer of larynx, ovarian cancer, cancer of the uterus, cancer of prostate, cancer of thyroid, brain tumor, Kaposi's sarcoma, angioma, leukemia, plethora vera, neuroblastoma, glioma retinae, myeloma, bladder tumor, sarcoma, osteogenic sarcoma, myosarcoma, cancer of skin, basal cell cancer, skin appendage carcinoma, skin metastatic cancer, skin melanoma and, however, is not limited to these examples.

The antitumor agent according to the present invention can also be used in combination with other antitumor agents and/or immunotherapic agents. Other antitumor agent includes mitomycin, adriamycin, cisplatin, vindesine, vincristine, cyclophosphamide, ifosfamide, bleomycin, peplomycin, or etoposide. On the other hand, other immunotherapic agents include a microorganism or cell wall skeleton component of a bacterium, an immunoreactive polysaccharide, a cytokine being natural type or obtained by a genetic engineering approach, or a colony-stimulating factor. The immunoreactive polysaccharide as described above includes lenthinan, schizophyllan and the like, the bacterial cell wall skeleton component includes muramyl dipeptide derivatives and the like, the microorganism includes a lactic acid bacterium and the like, and the cytokine being the natural type or obtained by the genetic engineering approach includes an interferon and the like.

When required, the antitumor agent according to the present invention can be added to the components as described above with components or additives used for preparation of a dug such as medical drugs, quasi-drugs and the like by free selection and combination in a range which does not damage an effect of the invention. The antitumor agent according to the present invention can be used by compounding it in medical drugs, quasi-drugs and the like in addition to a use as a single drug.

The form of the antitumor agent according to the invention is not specially restricted, but the appropriate form most suitable for a purpose can be selected from drug forms for oral administration or parenteral administration.

The drug preparation form suitable for oral administration includes, for example, a tablet, capsule, powder, drink, granule, fine granule, syrup, solution, emulsion, suspension, chewable and the like. The drug preparation form suitable for parenteral administration includes, for example, injection (subcutaneous injection, intramuscular injection, intravenous injection and the like), external use, drip, inhalation, spray and the like and, however, is not restricted to these forms.

A liquid drug preparation, for example, solution, emulsion or syrup, which is suitable for oral administration, can be prepared by using water, saccharides such as sucrose, sorbit and fructose, glycols such as polyethylene glycol and propylene glycol, oils such as sesame oil, olive oil and soybean oil, antiseptic agents such as p-hydroxy benzoic acid esters, and flavors such as strawberry flavor and peppermint. On the other hand, solid drug preparation, for example, tablet, capsule, powder, granule and the like, can be prepared by using an excipient such as lactose, glucose, sucrose and mannite, a disintegrating agent such as starch and sodium alginate, a lubricant such as magnesium stearate and talc, a binder such as polyvinyl alcohol, hydroxy propyl cellulose and gelatin, a surfactant such as fatty acid ester, a plasticizer such as glycerin.

The drug preparation for injection or drip suitable for parenteral administration includes preferably the material, which is the active ingredient, as described above in a sterilized water-based medium, which is isotonic to blood of a recipient, in a dissolved or suspended condition. For example, in case of the injection, the solution can be prepared by using a water-based medium and the like composed of a salt solution, a glucose solution, or the mixture of the glucose solution with the salt solution. The drug preparation for intestinal administration can be prepared by using a carrier such as cacao butter, hydrogenated fat or hydrogenated carboxylic acid, and can be used as a suppository. In addition, for preparation of spray, a carrier which allows the material being the active ingredient as described above to disperse as fine particles, does not irritate a mouth cavity and an air way mucosa of the recipient, and makes absorption of the active ingredient easy, can be used. The carrier is specifically exemplified by lactic acid, glycerine and the like. In accordance with a property of the material being the active ingredient and the carrier to be used, the drug preparation having forms such as an aerosol or dry powder. These preparations for parenteral administration are also added with 1 or 2 or more species of eatables and drinkables selected from glycols, oils, flavors, antiseptic agents, excipients, disintegrating agents, lubricants, binders, surfactants, plasticizers or the like.

The dose and administration frequency of the antitumor agent according to the present invention can be properly selected in accordance with various factors including a purpose of administration, a form of administration, conditions of a recipient such as an age, body weight and sexuality and, however, as a rule, the amount of administration of the active ingredient ranges from 1 to 10000 mg/kg/day, preferably 10 to 2000 mg/kg/day, more preferably 10 to 200 mg/kg/day. It is preferable to administer the preparation of the amount as described above in 1 to 4 frequencies a day. The time of administration of the antitumor agent according to the present invention is not specially restricted.

The invention also relates to food and drink which comprises the mixture of lactic acid oligomers as mentioned above. Namely, the mixture of lactic acid oligomers used in the invention can be not only used in the forms of a single preparation as described above, but also used by compounding it in food and drink.

A compounding form of food and drink according to the invention is not specially restricted, when it is satisfied to compound the mixture of lactic acid oligomers without decomposition.

The product of food and drink according to the present invention includes specifically a health food or a supplementary food including beverages, which is generally called a refreshing drink, drink agent, health food, specified health food, functional food, function activating food, nutriceutical food, supplement, feed, feed additive and the like. The antitumor agent according to the present invention can be used as a veterinary drug, feed and the like.

Food and drink include arbitrary food and dinks, and examples thereof include confectionaries such as chewing gum, chocolate, candy, tablet confectionary, jelly, cookie, biscuit and yogurt, cold confectionaries such as ice cream and ice confectionary, beverages such as tea, refreshing drink (including juice, coffee, cocoa and the like), nourishment drink agent and esthetic drink agent, bread, ham, soup, jam, spaghetti, frozen foods. Alternatively, the mixture of lactic acid oligomers according to the present invention can also be used by adding it to a flavoring material or a food additive. By taking food and drink according to the present invention, the antitumor effect is obtained to provide safe food and drink which show no substantially harmful adverse effect.

The food and drink according to the present invention can be obtained by directly mixing and dispersing the mixture of lactic acid oligomers to a common material used for foods and then processing the same in a desired form by a publicly known method.

The food and drink according to the present invention encompasses food and drink in every form, and the types are not specifically limited. That is, the food and drink can be provided by mixing the antitumor agent of the present invention into the above-mentioned various food and drink, or various nutrient compositions, such as various oral or enteral nutrient preparations or drinks. Compositions of such food and drink may include protein, lipid, carbohydrate, vitamin and/or mineral, in addition to the mixture of lactic acid oligomers. The form of the food and drink is not specifically limited, and may be in any form, such as solid, powdery, liquid, gel, and slurry forms, so far as it is in a form that is easily ingested.

The content of the mixture of lactic acid oligomers in the food and drink is not specifically limited, and is generally 0.1 to 20 weight %, more preferably approximately 0.1 to 10 weight %.

The mixture of lactic acid oligomers is preferably contained in the food and drink in an amount which achieve an antitumor effect which is an object of the present invention. Preferably, about 0.1 g to 10 g, more preferably about 0.5 g to 3 g, of the mixture of lactic acid oligomers is contained per food or drink to be ingested.

The present invention is further described in the following examples, but the scope of the present invention is not limited by the examples in any way.

### EXAMPLE

### Synthesis Example 1

Production of a Mixture of Poly Lactic Acids (Hereinafter Referred To As X01)

500 ml of L-lactic acid (to which D-lactic acid was also mixed) was placed into a separable flask in a mantle heater. 300 ml/min of nitrogen gas was flowed therein while stirring. Accumulated water was introduced into a flask equipped with a reflux condenser via a warmed descending type connecting tube, while heating at 145° C. for 3 hours. Furthermore, after pressure was reduced to 150 mmHg and heated at the same temperature for 3 hours, the mixture was heated at 155° C. for 3 hours under a reduced pressure of 3 mmHg, and then at 185° C. for 1.5 hours under a reduced pressure of 3 mmHg to obtain poly lactic acids as a reaction product.

The obtained poly lactic acids were kept at 100° C., and 100 ml of ethanol and 400 ml of methanol were separately added thereto, and then the mixture was allowed to be cooled. This mixture was added to 500 ml of methanol, and the mixture was well stirred and left to stand. Then, the mixture was filtrated for purification. The filtrate was subjected to vacuum drying and then dissolved in acetonitrile to obtain 200 ml (stock solution) in total.

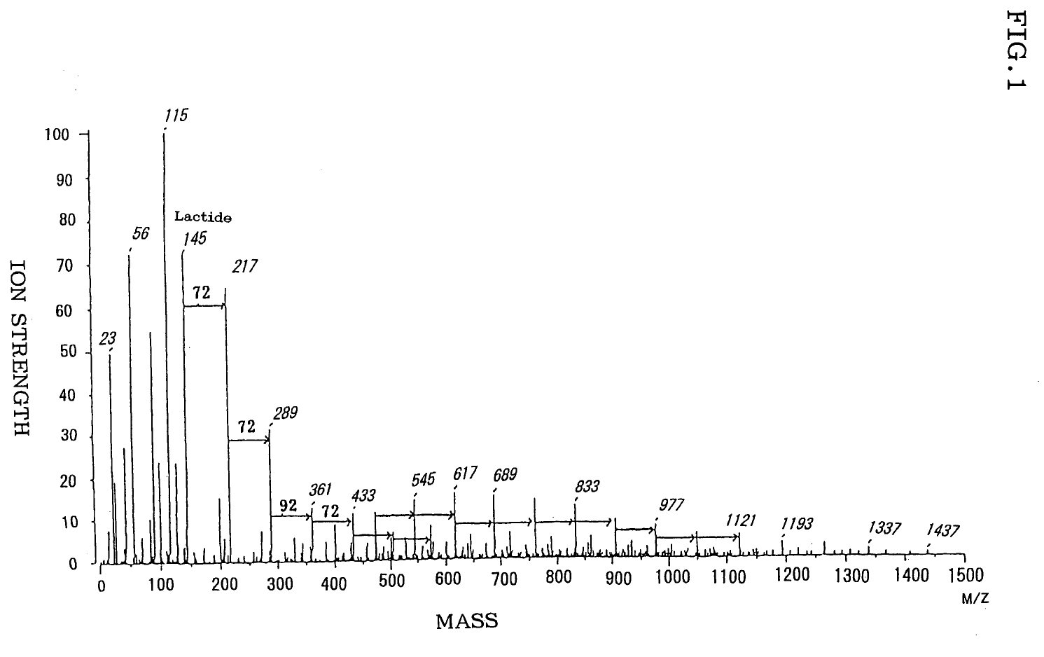

The stock solution was subjected to a reverse phase ODS column (TSK gel ODS-80™) which was previously equilibrated, and was stepwise eluted with 30%, 50% and 100% acetonitrile (pH2.0) each containing 0.01M hydrochloric acid to obtain poly lactic acids (condensation degree of 3 to 20) as an acetonitrile 100% elution fraction. The mass spectrum of the obtained substance is shown in FIG. 1. As is clear from the regular fragment ion peaks in FIG. 1, the obtained mixture of poly lactic acids comprises cyclic condensate and a linear condensate in a mixed state.

### Synthesis Example 2

Production of a Mixture of Cyclic Polylactic Acids (Hereinafter Referred To As X02)

10.0 g of (s)-(+)-lactic acid was placed in an eggplant-shaped flask having a inner volume of 100 ml, and was loaded on a rotary evaporator. The internal pressure of the flask was regulated to a range from 350 to 400 mmHg, and the mixture was heated to 140° C., followed by continuing the reaction at this temperature and this pressure for 6 hours (first heating step). Byproduct water produced by the reaction was removed by distillation. In the reaction condition as described above, lactide was almost not removed out from the system.

Subsequently, the reaction temperature was increased to 150 to 160° C. and the reaction pressure was gradually dropped from 400 mmHg over 6 hours to make finally 15 to 20 mmHg (pressure-dropping rate: 1 mmHg/min). Under the condition of this pressure-dropping rate, byproduct water was removed by distillation and, however, lactide was almost not removed out. Thereafter, the pressure was maintained at 15 to 20 mmHg to continue the reaction for 6 hours (second heating step).

Next, the pressure was dropped to 1 to 3 mmHg over 30 minutes, and the reaction was continued at 160° C. for 5 hours (third heating step).

After completion of the reaction as mentioned above, the reaction product was analyzed resulting in the yield of the cyclic oligomer of 6.80 g (yield 85%) having 3 to 21 of average polymerization degree.

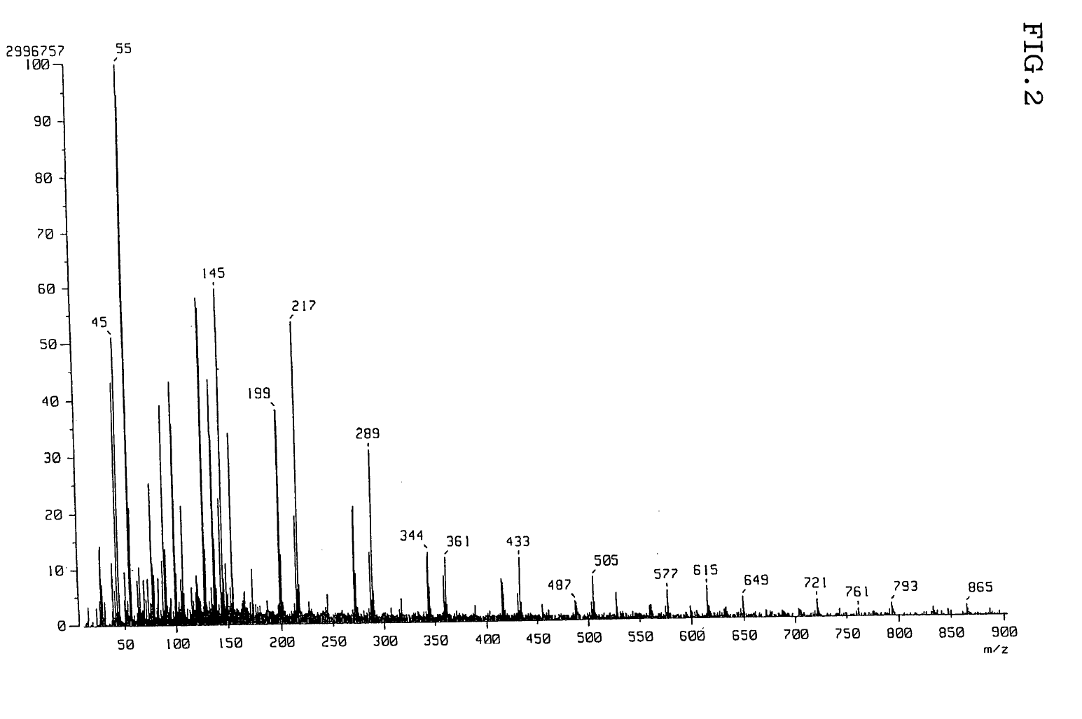

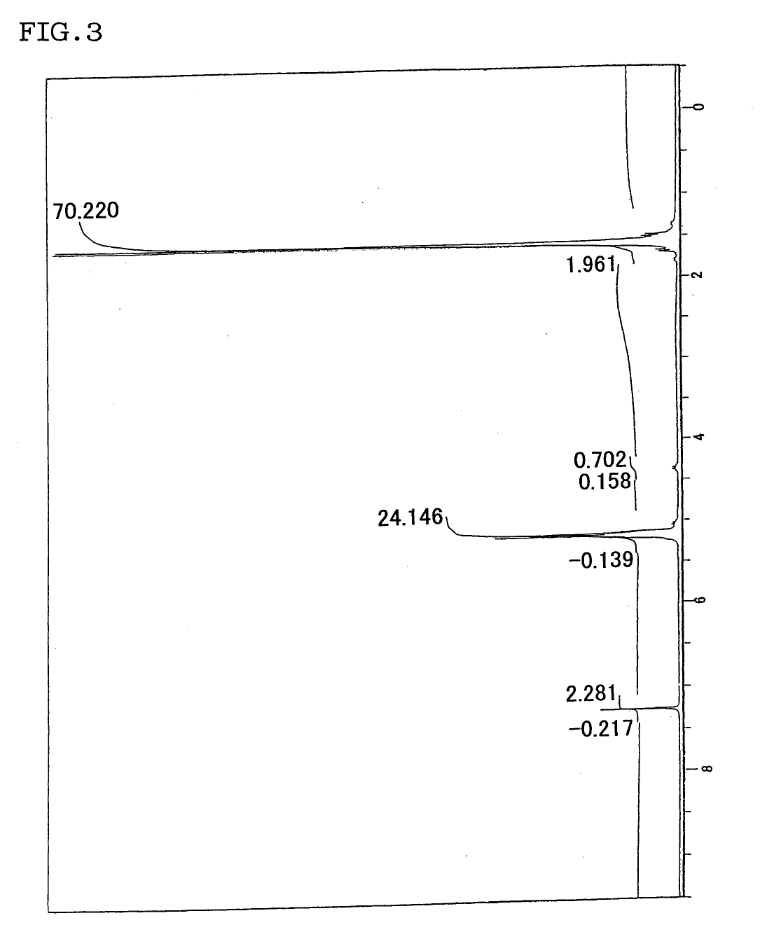

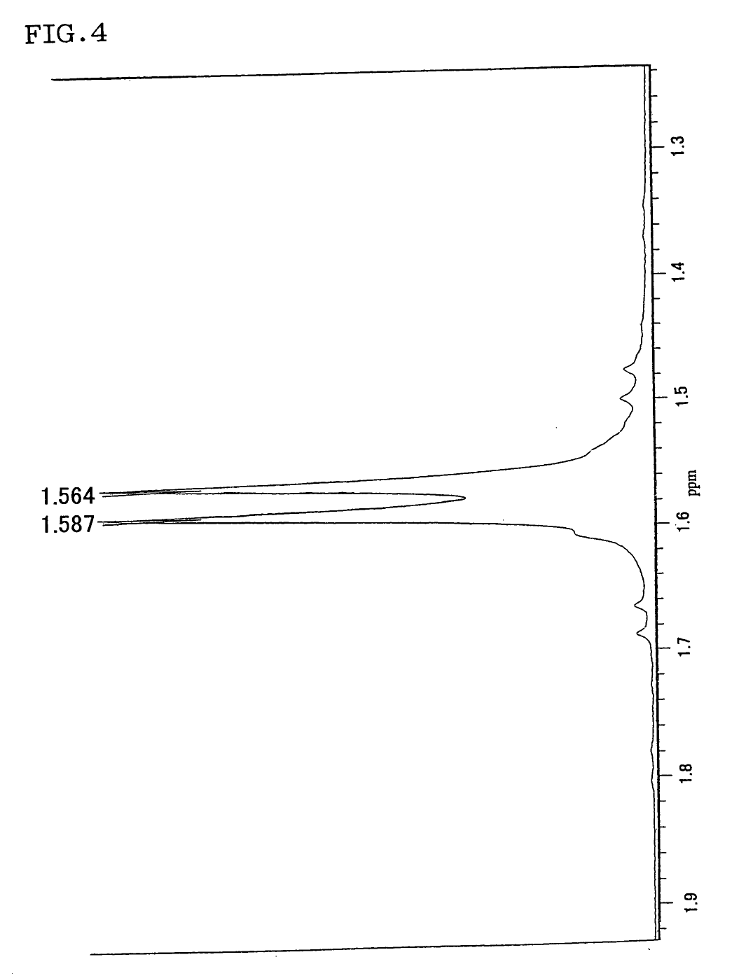

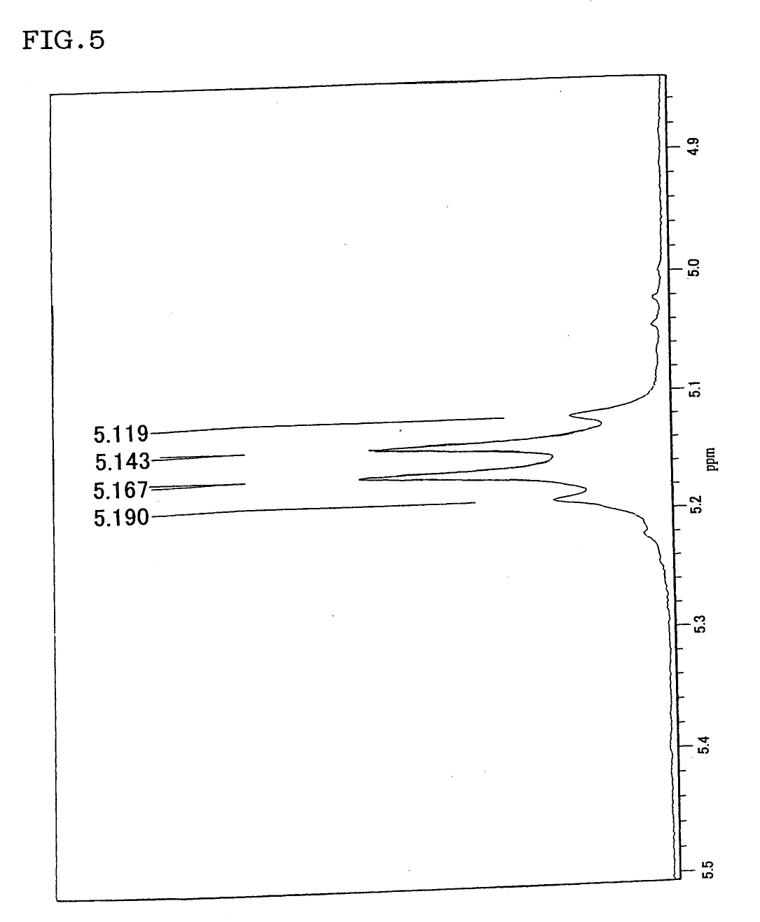

FIG. 2 shows the MS spectrum of the reaction product obtained in Synthesis Example 2. In addition, FIG. 3 shows the whole view of the NMR of the reaction product obtained in Synthesis Example 2. FIGS. 4 and 5 show the enlarged views of parts of FIG. 3.

### Synthesis Example 3

Production of a Mixture of Lactic Acid Oligomers (Hereinafter Referred To As X03)

The reaction scheme of Synthesis Example 3 is shown below.

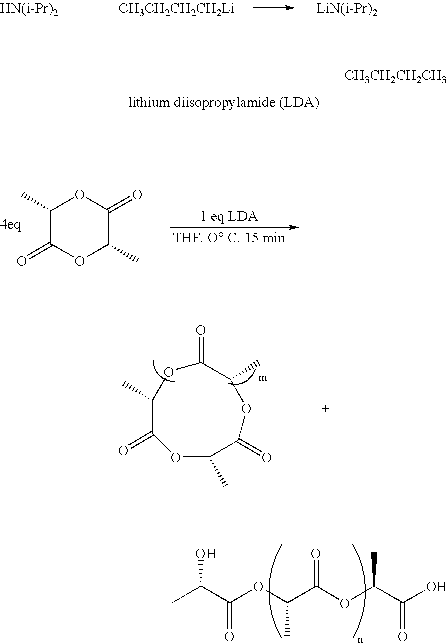

0.63 ml of n-butyllithium (1.6 M hexane solution, 1 mmol) was added to a 5 ml THF solution containing 0.101 g (1 mmol) of diisopropylamine at 0° C. under a nitrogen gas atmosphere, and the obtained mixture was stirred for 10 minutes, so as to obtain lithium diisopropylamide (LDA). Thereafter, 4 ml of THF solution containing 0.577 g (4 mmol) of L-(−)-lactide was added thereto followed by stirring for 15 minutes for reaction. Thereafter, 20 ml of a saturated ammonium chloride aqueous solution was added to the obtained reaction mixture to treat the reaction, and 10 ml of water was further added thereto. Extractions were carried out 5 times with THF (50 ml), and the organic layer was dried with anhydrous sodium sulfate. After anhydrous sodium sulfate was filtrated, the organic solvent was subjected to vacuum concentration, so as to obtain 0.53 g of a crude product. 6 ml of ether was added to the obtained crude product, and the mixture was immersed in an ultrasonic cleaner for 10 minutes for filtration, so as to obtain 0.39 g of a white solid product having a melting point of 125° C. to 129° C.

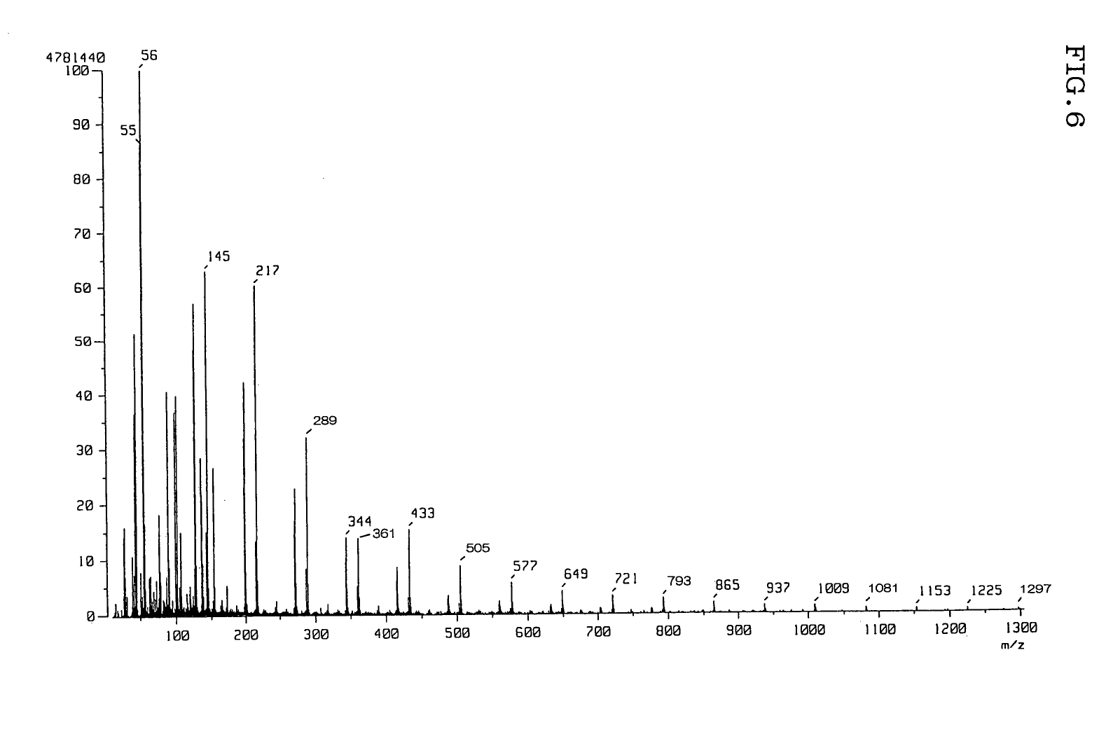

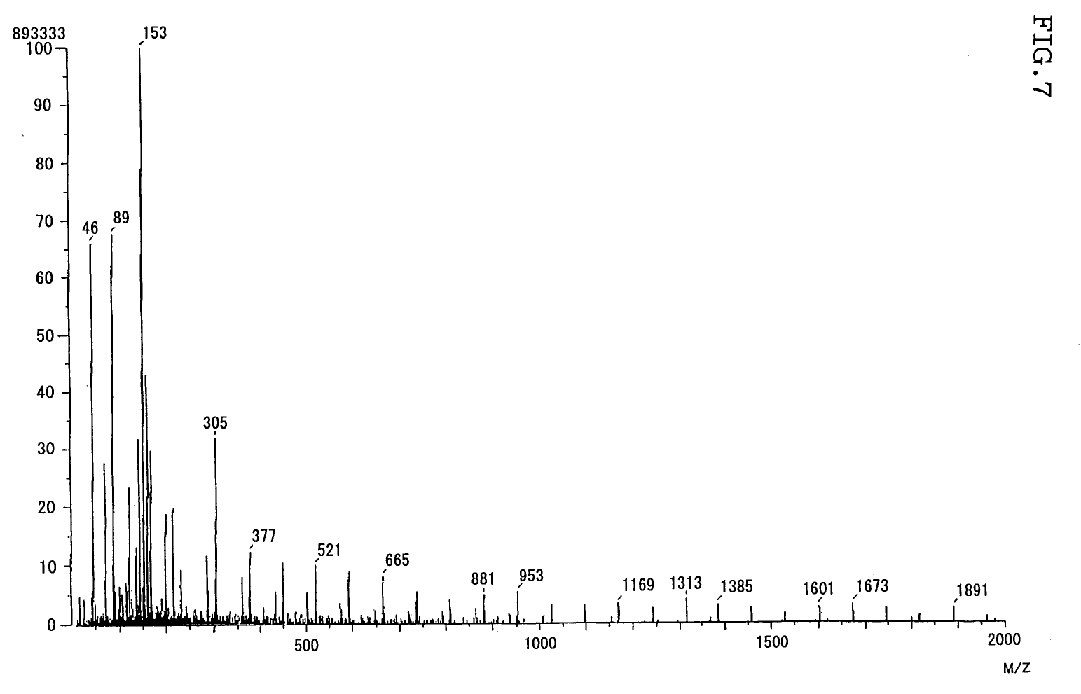

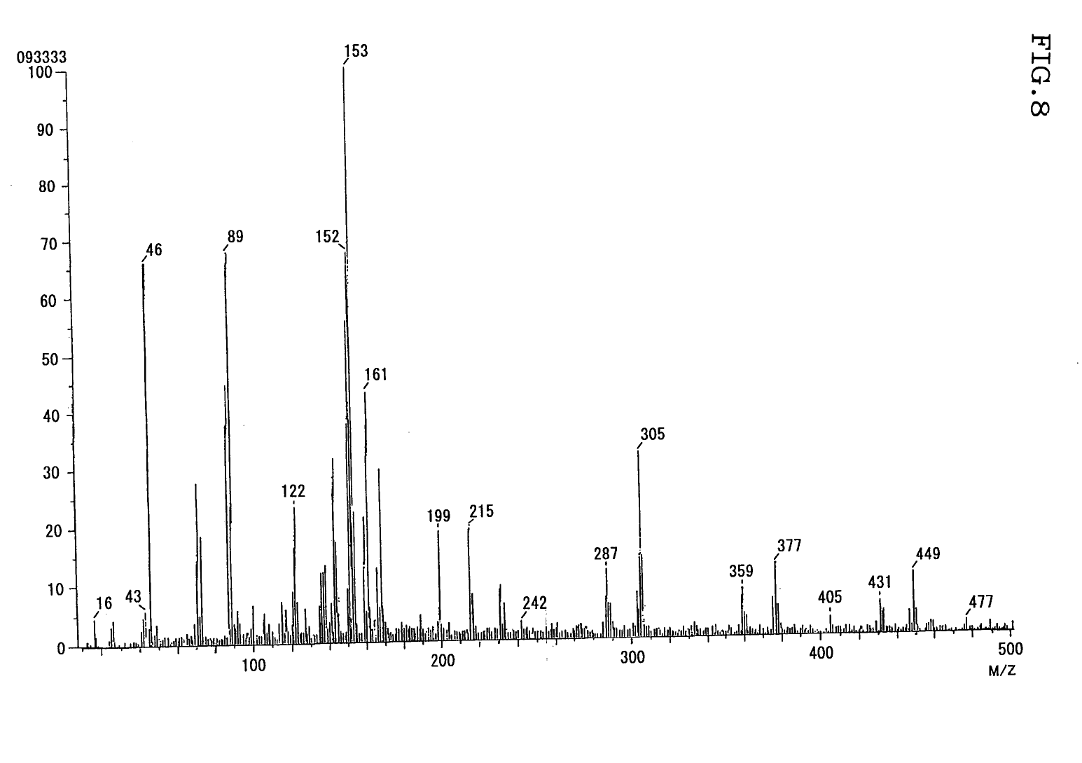

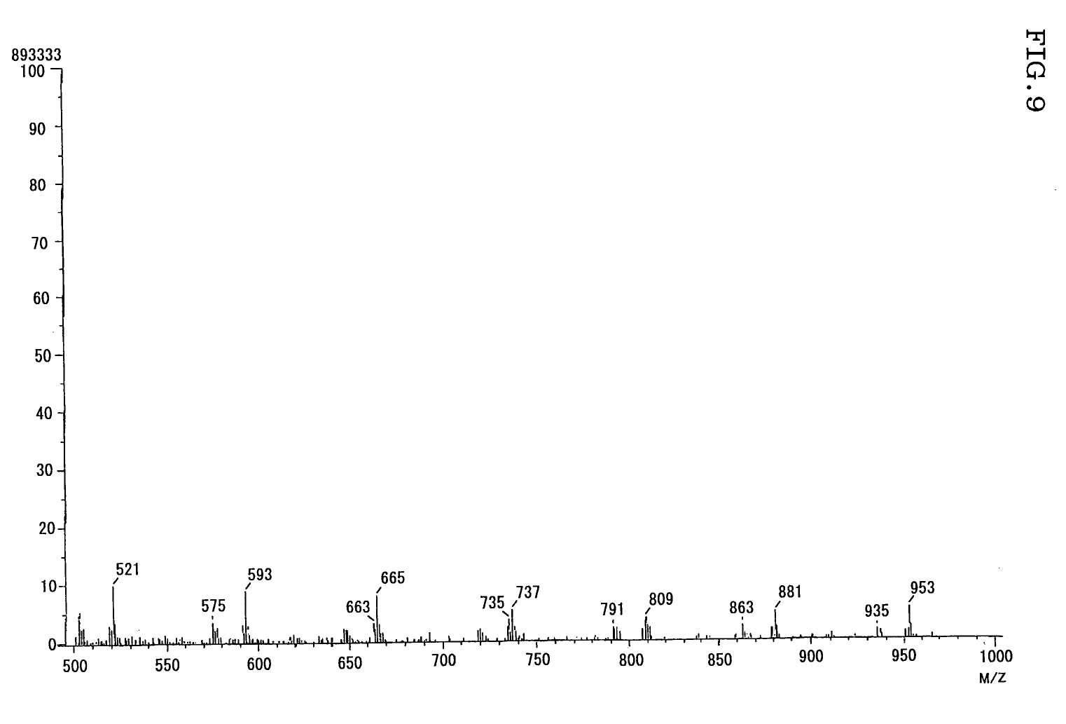

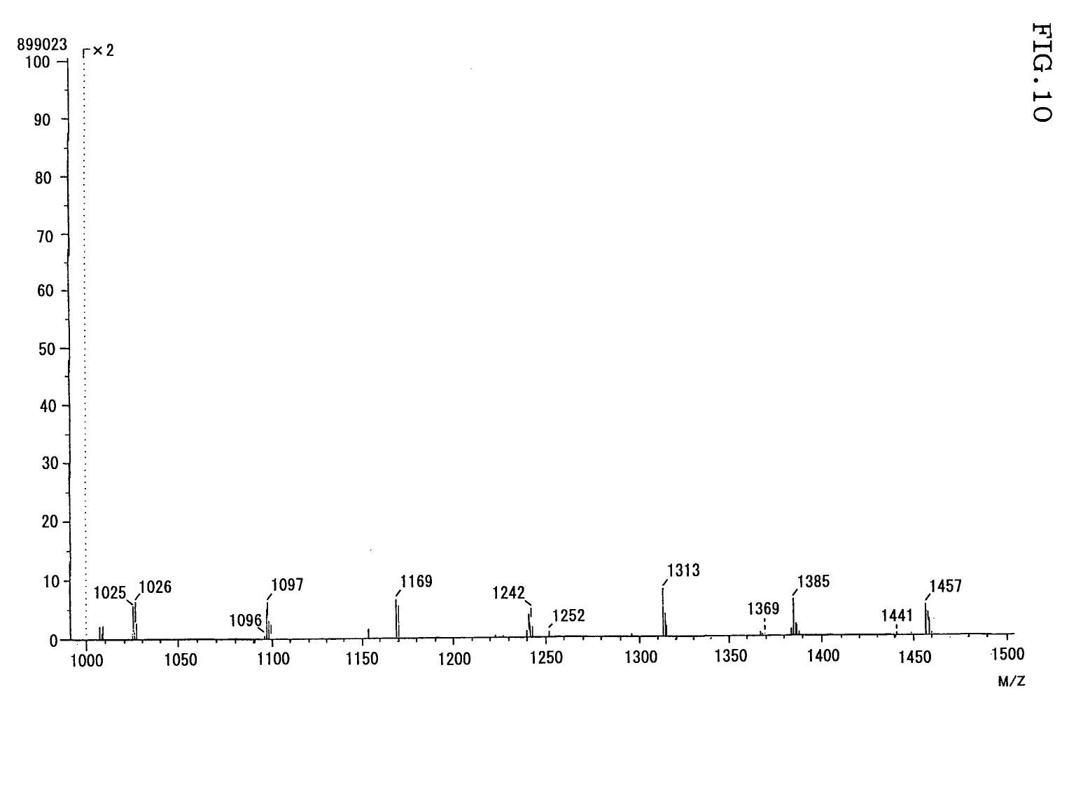

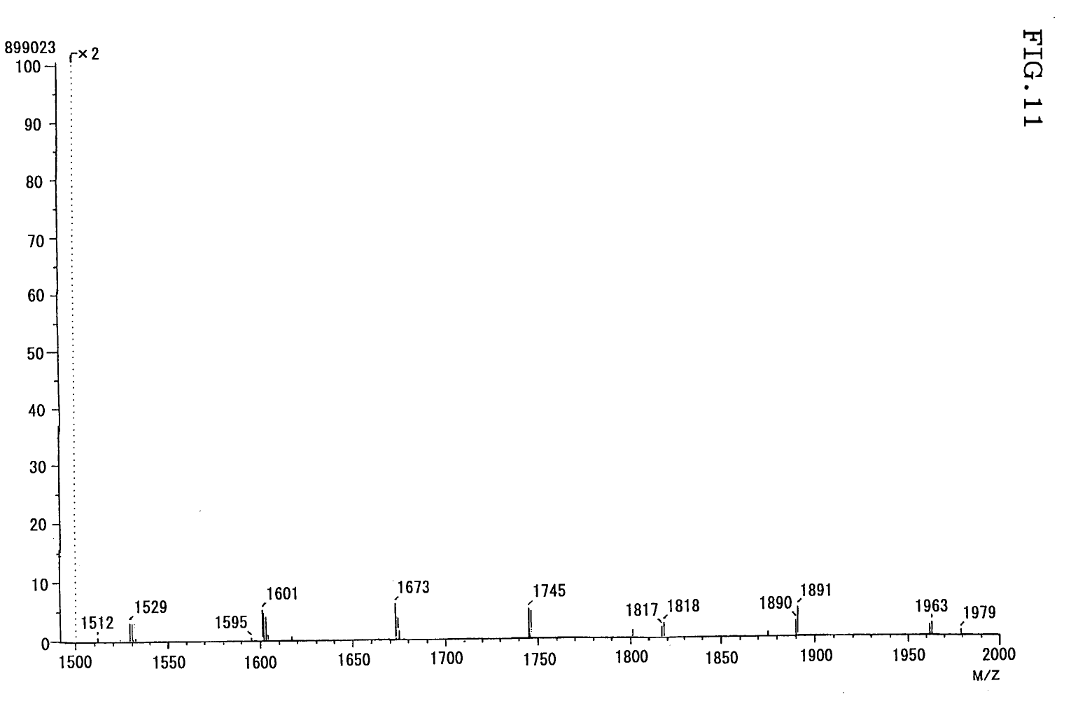

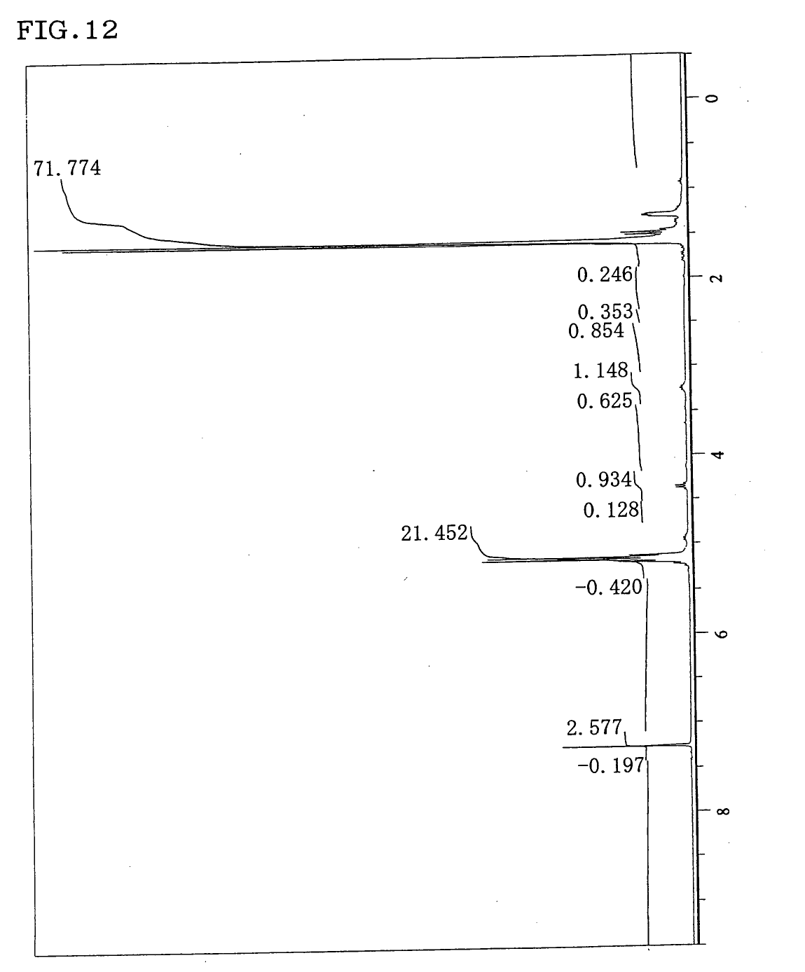

The physical data of the obtained product are shown in FIGS. 6 to 12. From the FABMS and NMR data shown in FIGS. 6 to 12, it was confirmed that a 3-mer to 21-mer cyclic lactic acid oligomer and a 3-mer to 27-mer linear lactic acid oligomer were present in the solid product.

### Test Example 1

(A) Material and Method

(1) Experimental Animal and Transplantation of Tumor Cells

Lewis lung cancer cells were transplanted at a quantity of 1×10⁴ cells into the muscle of the right femoral region of 9-week-old female mice (C57BU6N).

(2) Administration of Test Substance

The mice were divided into a control group (solvent administration group), an X01 administration group, an X02 administration group, and an X03 administration group. X03 was administered orally or abdominally. When X03 was administered via an oral route, 0.1% powder-mixed feed was given to the mice. On the other hand, when it was administered via an abdominal route, 1.0 mg of the test substance was administered per mouse every other day. Administration of the test substance began on the 2^{nd} day after the transplantation, and the administration was continued until euthanasia was performed on the mice on the 17^{th}, 18^{th} or 19^{th} day after the transplantation.

(3) Histological Studies

Spongy tissues located in the center of excised tumor tissues were removed therefrom. Thereafter, the tissue section was sliced and fixed. After dehydration, it was embedded in a hydrophilic methacryl resin. A section was prepared from the embedded tissue block, and it was then subjected to the H-E staining, followed by observation.

(B) Results and Conclusions

(1) Concerning Tumor Weight

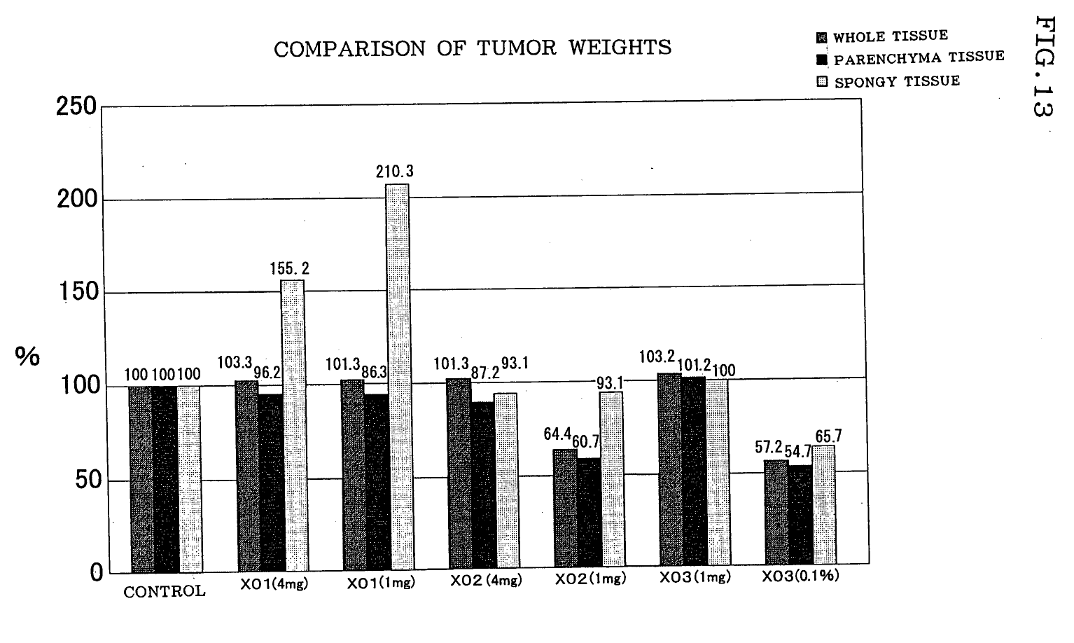

FIG. 13 shows the measurement results of the tumor weight. When X03 was administered via an oral route, the effect of inhibiting the growth of tumor tissues was significantly recognized, as in the case of administration of X02 via an abdominal route (FIG. 13). On the other hand, in the case of X01, there were no significant differences in the weight of a tumor itself between the case of oral administration and the case of abdominal administration, and spongy tissues are remarkably increased.

(2) Concerning Lung Metastasis Colonies and Tumor Weight

Lung metastasis colonies range from a large colony having a diameter of 2 mm or greater to a very small colony that is the size of a needlepoint. As the number of colonies is increased, huge colonies are likely to be increased. FIG. 15 shows the relationship between the total number of colonies and the tumor weight. The number of colonies was significantly decreased in the cases of a 4-mg X01 abdominal administration group, a 1-mg X02 abdominal administration group, a 1-mg X03 abdominal administration group, and a 1-mg X03 oral administration group (0.1%). Repression of tumor weight was apparently observed in X02 and X03 administration groups, and it was not observed in an X01 administration group.

(3) Conclusion

The following Table 1 shows the above-described results and results obtained from the observation of neutrophils. When compared with a control, those having a certain effect are represented with the symbol ◯, those having an excellent effect are represented with the symbol ⊚, those that did not have a significant effect are represented with the symbol Δ, and those having no effects are represented with the symbol X. In the case of X03, neutrophils were infiltrated into tumor tissues, and it was found to have the highest lung metastasis inhibiting effect.

TABLE 1

| | | Frequency | |
|---|---|---|---|
| | Repression of | of appearance | Repression of |
| | tumor weight | Of neutrophils | lung Metastasis |
| X01 | X | Δ | ◯ |
| (4 mg, abdominal) | | | |
| X02 | ◯ | ◯ | ◯ |
| (1 mg, abdominal) | | | |
| X03 | ◯ | ◯ | ⊚ |
| (0.1%, oral) | | | |

### INDUSTRIAL APPLICABILITY

The present invention makes it possible to provide a novel antitumor agent and also provide food and drink using the same. Further, the mixture of polylactic acids used as an active ingredient in the present invention is a lowly condensed compound of a lactic acid derived from a component of a living body and, therefore, shows a high biocompatibility and a less adverse effect.

1. An antitumor agent which comprises a mixture of linear and cyclic lactic acid oligomers represented by the following formula (1) or (2): 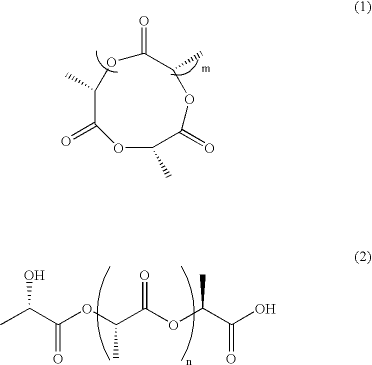
wherein m represents an integer of 1 to 30, and n represents an integer of 1 to 30,
which is produced by polymerizing lactides in the presence of a compound represented by the following formula (3):
Me—N(R¹)(R²) (3)
wherein Me represents an alkali metal, and each of R¹ and R² independently represents an aliphatic group or aromatic group.

2. The antitumor agent according to claim 1 wherein Me is lithium in the formula (3).

3. The antitumor agent according to claim 1 wherein each of R¹ and R² independently represents an alkyl group containing 1 to 6 carbon atoms in the formula (3).

4. The antitumor agent according to claim 1 wherein Me is lithium, and each of R¹ and R² is an isopropyl group in the formula (3).

5. The antitumor agent according to claim 1 wherein m is an integer of 1 to 19 in the formula (1).

6. The antitumor agent according to claim 1 wherein n is an integer of 1 to 25 in the formula (2).

7. Food and drink which comprise the antitumor agent according to claim 1.
